(19) United States
(12) Patent Application Publication
(10) Pub. No.: US 2025/0163483 A1
(43) Pub. Date: May 22, 2025

(54) PROCESS FOR PREPARING HIGH PURITY GALACTO-OLIGOSACCHARIDE

(71) Applicant: FrieslandCampina Nederland B.V., Amersfoort (NL)

(72) Inventors: Linqiu Cao, Wageningen (NL); Marlies Versteeg, Wageningen (NL)

(21) Appl. No.: 18/841,340

(22) PCT Filed: Feb. 23, 2023
(86) PCT No.: PCT/EP2023/054512

§ 371 Date: Aug. 23, 2024

(30) Foreign Application Priority Data
Feb. 25, 2022 (EP) 22158755.3

(51) Int. Cl. C12P 19/12; C12N 9/38; C12P 19/14

(52) U.S. Cl. CPC C12P 19/12; C12N 9/2471; C12P 19/14; C12Y 302/01023

(57) ABSTRACT

The invention relates to an enzymatic process for reducing the lactose content of a galacto-oligosaccharide composition and the production of a high purity galacto-oligosaccharide with a high retention of bifidogenic disaccharides.

The present invention relates to a high purity galacto-oligosaccharide (GOS) composition and a process for obtaining such composition.

Galacto-oligosaccharides comprise a chain of galactose units and a terminal glucose unit, that arises through consecutive transgalactosylation reactions catalyzed by a beta-galactosidase. Typical GOS compositions mainly comprise di- to hexa-saccharides. Some of the GOS components exist naturally in human breast milk and bovine colostrum.

Various physiological functions of GOS have been reported, including the capacity to stimulate the growth of bifidogenic bacteria in the gut, support normal gut transit, contribute to natural defenses, enhance mineral absorption, and stimulate immune functions and lower inflammations. GOS has received particular attention due to its prebiotic effects that promote the growth of Bifidobacterium, Lactobacillus, and other enteric bacteria. Therefore, GOS is used in infant formula, beverages fermented by Lactobacillus, yogurts, juices and drinks. Some of these GOS-containing foods are certified as Food for Specified Health Uses by the Consumer Affairs Agency in Japan, and GOS is certified as Generally Recognized As Safe (GRAS) substances by the U.S. Food and Drug Administration (GRAS Notices: GRN 233, 236, 285, 286, 334, 484, 489, 495, 518, and 569).

Study has shown that certain disaccharide GOS components (components with a degree of polymerization of 2; i.e. DP2 components), in particular gal-β1,2-glc and gal-β1,3-glc, are able to stimulate the production of mucin, which is critical for the colonization of bifidobacteria in the colon. Furthermore, in vitro fermentation studies have shown that these DP2 species are digested very fast, thereby suggesting that these DP2 GOS components are very bifidogenic and have high growth stimulating effect on bifidobacteria (Lammerts van Bueren et al., (2017) Scientific Reports 7:40478) GOS is conventionally made by contacting a lactose-containing feed with a beta-galactosidase enzyme. The resulting GOS is a mixture comprising galacto-oligosaccharides with different degrees of polymerization (DP) and lactose.

A large part of the world-wide population above 3 years of age suffers from lactose intolerance, which may result in abdominal pain, bloating, diarrhea, gas, and nausea upon consumption of lactose-containing compositions. Conventional GOS compositions contain large amounts of lactose and thus may cause these symptoms. For instance, commercial GOS syrups, such as Vivinal® GOS, often contain about 60 wt % oligosaccharides, 13-16 wt % lactose, and 20-21 wt % monosaccharides (e.g. 19-20 wt % glucose and 1.5 wt % galactose).

Therefore, a need exists to produce a GOS from which lactose has been largely removed.

There are various known ways to reach this goal. For instance, fermentation by yeasts (Pezmendi et al., Yeast; 2020:37:515-530) or strains of S. thermophilus (WO 2011/016008) or Kluyveromyces lactis (EP3205727, CN111334541, and CN107523595).

Disadvantages of fermentation processes are (i) their relatively low productivity due to the required low substrate concentrations, (ii) the required addition of nutrients for the growth of the microorganisms, resulting in the presence and formation of contaminants and/or side products such as ethanol or glycerol, and (iii) the potential formation of allergens.

Another way to remove lactose from GOS is enzymatic lactose removal from an existing GOS composition using a lactase enzyme.

This method has been applied by WO 2017/120678 and WO 2019/119102, which use a yeast lactase, preferably derived from Kluyveromyces lactis. The enzyme is used in a concentration of 1-50 LU/g lactose and a temperature of 30-45° C.

The GOS to be purified according to these disclosures was obtained by using a beta-galactosidase obtained from Aspergillus oryzae and this GOS had a high content of lactose and a low content of other disaccharides. Although the content of disaccharides other than lactose in this GOS composition was largely retained upon lactose hydrolysis, this is not surprising since the initial lactose content was very high, the disaccharide/lactose ratio was low, and the lactose concentration remained above 8 wt % during the reaction. In other words, there was always enough lactose available for the enzyme so that it did not have to use other disaccharides as substrate.

A problem associated with this type of enzymatic reactions is that when the lactose concentration becomes very low, e.g. 1-2 wt %, the other disaccharide components, especially gal-β1,2-glc and gal-β1,3-glc, will become good substrates for the lactase enzyme, thereby resulting in hydrolysis of these species and a consequential loss of bifidogenic effects.

It has now been found that, by selecting the right conditions, a GOS composition with a lactose content below 6 wt %, based on total carbohydrate weight and a relatively high content of gal-β1,2-glc and gal-β1,3-glc can be obtained.

The present invention therefore relates to a process for reducing the lactose content of a galacto-oligosaccharide composition. This process comprises the steps of:
a) providing an aqueous solution of a galacto-oligosaccharide composition with a lactose content of 10-30 wt %, based on total carbohydrate weight, and a weight ratio of (gal-β1,2-glc+gal-β1,3-glc):lactose in the range 1.5:1 to 3.0:1, said solution have a dry matter content of at most 54 wt %,
b) adding to this solution a lactase enzyme,
c) conducting an enzymatic reaction at 30-50° C. to allow hydrolysis of lactose towards monosaccharides until the lactose content of the solution is reduced to at most 6 wt % of lactose, based on total carbohydrate content, and
d) removing monosaccharides from the solution obtained in step c).

The lactose content of the resulting GOS composition will be not more than 6.0 wt %, based on total carbohydrate weight.

The invention also relates to a GOS composition comprising, based on total carbohydrate weight, at least 90 wt % of oligosaccharides, 10-40 wt % of the disaccharides gal-β1,2-glc and gal-β1,3-glc, and less than 6 wt % of lactose.

The oligosaccharide content, lactose content, the content of said disaccharides, and the total carbohydrate content is determined by HPAEC-PAD, as disclosed by S. van Leeuwen et al, Carbohydrate Research 425 (2016) 48-58, and in the examples below.

The starting GOS composition, with the required lactose content and (gal-β1,2-glc+gal-β1,3-glc):lactose ratio, can have been prepared by various beta-galactosidase enzymes, such as those produced in the microorganisms Bacillus circulans, Kluyveromyces fragilis, Sporobolomyces singularis, Lactobacillus fermentum, Papiliotrema terrestris, Bifidobacterium bifidum, and Lactobacillus bulgaris. In contrast to, for instance, beta-galactosidase from Aspergillus oryzae, these enzymes enable the formation of the right lactose and bifidogenic disaccharide levels to enable the retention of said disaccharides in the present process.

Preferred enzymes are those produced by Bacillus circulans or Papiliotrema terrestris. A highly preferred enzyme is a beta-galactosidase produced by Papiliotrema terrestris; not only does it provide the right lactose and bifidogenic disaccharide levels to enable the retention of said disaccharides in the present process, the resulting GOS is also hypoallergenic in the sense that the resulting GOS composition does not cause elevated reaction in the Basophil activation test.

GOS production leads to a GOS syrup. The lactose content of such GOS should be in the range 10-30 wt %, based on carbohydrate content. The weight ratio (gal-β1,2-glc+gal-β1,3-glc):lactose in said GOS syrup should be in the range 1.5:1 to 3.0:1.

The dry matter content of such syrup is generally in the range 40-75 wt %.

According to the process of the present invention, an aqueous solution of the GOS composition to be purified is contacted with a lactase enzyme.

This GOS solution may be a (diluted) syrup resulting from the GOS preparation process or it may be dissolved GOS powder. Any enzyme used in the GOS preparation process may have been inactivated and optionally removed, or may still be in active form at the start of the process of the present invention. This optional deactivation can be conducted by adjusting the pH to 2 or less, for instance by adding HCl, or by heating to, e.g., 95° C.

If not already within this concentration range, the dry matter content of the GOS solution should be adjusted to at most 54 wt %, preferably at most 50 wt %, more preferably in the range 40-50 wt %, most preferably in the range 43-48 wt %.

The lactose content of the starting GOS solution is in the range 10-30 wt %, preferably 10-25 wt %, more preferably 10-20 wt %, based on total carbohydrate dry weight.

The weight ratio (gal-β1,2-glc+gal-β1,3-glc):lactose in the GOS solution is in the range 1.5:1 to 3.0:1, preferably 1.5:1 to 2.5:1, more preferably 1.5:1 to 2.0:1.

The GOS solution preferably has a pH of 5.5-7.5, more preferably 6.0-7.0, most preferably 6.3-6.8. The pH can be regulated by a food grade buffer, such as a citrate or phosphate buffer, and with 10 mM KCl and 2.5 MgCl₂, preferably in a concentration of 5 mM-20 mM.

The enzyme is a lactase enzyme, preferably selected from the lactase enzymes produced by Kluyveromyces lactis (e.g. Maxilact® 5000, ex-DSM), Bifobacterium bifidum (e.g. Nola@Fit, ex-Chr. Hansen), and Lactobacillus bulgaris (e.g. Bonlacta™ ex-IFF). A particularly preferred enzyme is a lactase enzyme derived from Kluyveromyces lactis.

The dosage of the lactase enzyme depends on the reaction temperature and reaction time; higher reaction temperature and/or longer reaction times allow lower enzyme concentrations.

The enzyme dosage, reaction time, and temperature should be chosen such that the lactose content is reduced to at most 6 wt % lactose, based on total carbohydrate weight, preferably at most 5.5 wt %, more preferably at most 5.0 wt %, even more preferably in the range 3.0-5.0 wt %, more preferably in the range 3.5-5.0 wt %, most preferably in the range 4.0-5.0 wt %.

The enzyme can be used in powder form (e.g. freeze dried, vacuum dried, or spray dried) or liquid form (e.g. dissolved in a phosphoric acid buffer solution, a tri-ethanol amine buffer solution, a tris-hydrochloric acid buffer solution, or a GOOD buffer solution).

In a specific embodiment, the enzyme is used in immobilized form. Various ways of enzyme immobilization are known in the art. They typically comprise a porous carrier onto which the beta-galactosidase is immobilized via covalent binding, via physical absorption (charge-charge or van der Waals interaction), via gel encapsulation, or a combination thereof. Besides, carrier-free immobilized enzymes such as CLEC (cross-linked enzyme crystals) or CLEA (crosslinked enzyme aggregates) might be also applied.

Carriers that can promote direct covalent binding of the enzyme are preferred, in view of their ease of operation and absence of leakage into the reaction mixture. An example of a solid carrier is an activated acrylic polymer, preferably a functionalized polymethacrylate matrix. For example, a hexamethylenamino-functionalized polymethacrylate matrix (Sepabeads) or a microporous acrylic epoxy-activated resin, like Eupergit C 250L, can be used.

The use of immobilized enzyme allows a repeated batch operating system involving several consecutive batches (‘cycles’) of GOS purification. It also allows for recycling of enzyme, which enables semi-continuous operation and multiple reuse of the enzyme.

The enzymatic reaction is then conducted at 30-50° C. preferably 35-45° C., most preferably 38-42° C. to allow hydrolysis of lactose to the required extent. Reduction of the lactose content to the desired level will generally take about 1-8 hours, preferably 2-6 hours, most preferably 3-5 hours.

At the end of the reaction, the enzyme may be deactivated by conventional methods, such as pH adjustment and/or temperature increase of the solution. For instance, the pH may be adjusted to about 4.5, and/or the temperature may be increased to about 72° C.

Lactose is hydrolyzed into its monosaccharides glucose and galactose, which monosaccharides can be removed from the GOS solution by conventional methods, such as nanofiltration or simulated moving bed chromatography (SMB), more preferably sequential simulated moving bed chromatography (SSMB). SSMB is the preferred method because SSMB usually enables high product recovery (98-99.5%) and high purity (97-99.5%). Moreover, SSMB requires less maintenance and lower water consumption than NF and consumes less solvent than non-sequential SMB. The (sequential) SMB chromatography will be based on size exclusion. Suitable resins for such size exclusion chromatography are ion-exchange resins and gel-type resins.

The so-purified GOS will have a lactose content of not more than 6 wt %, preferably not more than 5.5 wt %, more preferably at most 5.0 wt %, even more preferably in the range 3.0-5.0 wt %, more preferably in the range 3.5-5.0 wt %, and most preferably in the range 4.0-5.0 wt %. based on total carbohydrate content.

The monosaccharide content will be below 6 wt %, preferably below 5.5 wt %, even more preferably below 5 wt %, more preferably below 4.5 wt %, and most preferably below 4 wt %.

The purified GOS will have a content of the disaccharides gal-β1,2-glc and gal-β1,3-glc in the range 10-40 wt %, preferably 15-30 wt %, based on oligosaccharide content.

The process according to the invention is able to retain 60-80% of these disaccharides. The final purified GOS composition preferably has a oligosaccharide/lactose ratio of more than 10, more preferably more than 15 and most preferably more than 20, meaning that it is considered as clinically lactose-free and will not cause any lactose-intolerance symptoms.

The resulting purified GOS can be added to or used as a nutritional composition or nutritional supplement. It can be used as aqueous solution/syrup, or it can first be dried, e.g. by spray-drying, freeze-drying, or spray-cooling, to form a powder.

The GOS composition can be administered to a subject in the form of a nutritional composition or food supplement. The subject is a mammal, in particular a human being. Although the subject may have any age, the subject is preferably aged at least 18 months, preferably at least 24 months, even more preferably at least 3 years (36 months), and most preferably at least 13 years. In a most preferred embodiment, the subject is an adult.

Such nutritional compositions or food supplements may contain, apart from the GOS composition, one or more further ingredients, such as protein sources, probiotics, lipid sources, probiotics, human milk oligosaccharides, and/or digestible carbohydrates. The nutritional composition may have a liquid, semi-liquid, or solid constituency. Examples of suitable forms are dairy products, such as milk, milkshake, chocolate milk, yoghurt, cream, cheese, pudding, and ice cream; bars, such as nutritional bars, energy bars, snack bars, cereal bars, and bars for diabetics; liquid products, such as nutritional drinks, diet drinks, liquid meal replacers, sports drinks, and other fortified beverages; savory snacks, such as chips, tortillas, puffed and baked snacks, crackers, pretzels, and savory biscuits; bakery products, such as muffins, cakes, and biscuits; sweets such as gummies and candies; and pastas, such as spaghetti.

Food supplements can have the form of pills, capsules, gummies, or dry powders. Food supplements may be ready for consumption or may need to be dissolved in a liquid like water. The product in dry powder form may be accompanied with a device, such as a spoon, to measure the desired amount of the powder (e.g. daily or unit dose). The nutritional composition may be provided in a jar, bottle, sachet, carton, rapping, and the like.

Examples of protein sources that may be present in the nutritional composition or food supplement include whey proteins (e.g. whey protein concentrate or whey protein isolate), casein (e.g. micellar casein isolate), milk protein concentrate or isolate, and/or plant proteins such as soy protein. In a preferred embodiment, the protein source is a hypoallergenic or non-allergenic protein source. This includes protein hydrolysates that can be administered to subjects having intolerance against dietary proteins, more particularly cow's milk proteins, without inducing allergic reactions. Examples of such protein hydrolysates are hydrolyzed whey proteins containing hydrolysis residues having a molecular weight below 10,000 Da and casein hydrolysate with peptides of maximally 3000 Da.

Examples of carbohydrate sources that may be present in the nutritional composition or food supplement are disaccharides such as saccharose, monosaccharides, such as glucose, and maltodextrins, starch and carbohydrate sources having a prebiotic effect. The presence of lactose is evidently undesired.

Examples of lipid sources that may be present in the nutritional composition or food supplement are tri-, di-, and monoglycerides, phospholipids, sphingolipids, fatty acids, and esters or salts thereof. The lipids may have an animal, vegetable, microbial or synthetic origin. Of particular interest are polyunsaturated fatty acids (PUFAs) such as gamma linolenic acid (GLA), dihomo gamma linolenic acid (DHGLA), arachidonic acid (AA), stearidonic acid (SA), eicosapentaenoic acid (EPA), docosahexaenoic acid (DHA), docosapentaenoic acid (DPA) and conjugated linoleic acid (CLA). CLA is important in the protection against eczema and respiratory diseases in children. This particularly involves the cis-9, trans-11 and cis-12 isomers of CLA. Examples of suitable vegetable lipid sources include sun flower oil, high oleic sun flower oil, coconut oil, palm oil, palm kernel oil, soy bean oil, etc. Examples of suitable lipid sources of animal origin include milkfat, for example anhydrous milkfat (AMF), cream, etc. In a preferred embodiment, a combination of milkfat and lipids of vegetable origin are used. Examples of probiotics that may be present in the nutritional composition or food supplement are (symbiotic) bacteria, such as Bifidobacteria and/or Lactobacillus. Further, the nutritional composition or food supplement may contain one or more conventional micro ingredients, such as vitamins, antioxidants, minerals, free amino acids, nucleotides, taurine, carnitine and polyamines. Examples of suitable antioxidants are BHT, ascorbyl palmitate, vitamin E, alpha and beta carotene, lutein, zeaxanthin, lycopene and phospholipids.

### EXAMPLES

### Example 1

GOS-containing solutions with dry matter contents as listed in Table 1 were obtained by diluting a Biotis™ GOS-O syrup with a dry matter content of 75 wt %. Said GOS syrup contained 13.2 wt % lactose based on total carbohydrate content and 24 wt % of gal-β1,2-glc and gal-β1,3-glc (presented as “DP2-GB” in Table 1) based on total carbohydrates.

In a 100 ml vial, the pH of the GOS solutions was adjusted to 6.5 using 10 mM sodium phosphate buffer, 2.5 mM MgCl₂ and 10 mM KCl.

Hydrolysis of the lactose was performed by adding Maxilact® 5000 (Kluyveromyces lactis, ex-DSM) to the solution in different amounts (see Table 1). The reaction mixture was heated with water bath at 40° C., while stirring with a magnetic stirrer. The reaction time was 4 hours.

After said four hour reaction time, a 1 ml sample was taken. To this sample, 1.5% (w/v) of a 1.5 M HCl solution was added and the resulting sample was heated at 95° C. for 20 minutes in order to denature the enzyme. After cooling down to room temperature, the sample was analyzed by HPLC using Dionex ICS-3000 workstation (equipped with a CarboPac PA-1 column (250×4 mm, Dionex) and an ICS-3000 ED pulsed amperometric detector (PAD), using a complex gradient of A: 100 mM NaOH, B: 600 mM NaOAc in 100 mM NaOH, C: Milli-Q water, and D: 50 mM NaOAc, as disclosed in S. van Leeuwen et al, Carbohydrate Research 425 (2016) 48-58. The fractionations were performed at 1.0 mL/min with 10% A, 85% C, and 5% D in 25 min to 40% A, 10% C, and 50% D, followed by a 35-min gradient to 75% A, 25% B, directly followed by 5 min washing with 100% B and reconditioning for 7 min with 10% A, 85% B, and 5% D.

As shown in Table 1, an enzyme dosage of 2.5 U/gram dry matter was not able to reduce the lactose content below 6 wt % lactose with 4 hours, whereas an enzyme dosage of 7.5 U/gram and dry matter contents of 55 wt % resulted in lower retention of DP2 oligosaccharides and lower selectivity for lactose hydrolysis than the other experiments.

TABLE 1

| | | | lactose | DP2-GB | Total DP2 | | |
|---|---|---|---|---|---|---|---|
| | Dry matter | Enzyme | removal | Retention | retention¹ | DP2-GB | Lactose |
| Exp. | (wt %) | (U/g dm) | (%) | (%) | (%) | (wt %) | (wt %) |
| 1 | 45 | 2.5 | 49.3 | 83.7 | 100.0 | 20.1 | 6.6 |
| 2 | 45 | 5.0 | 66.7 | 72.1 | 100.0 | 17.3 | 4.3 |
| 3 | 45 | 7.5 | 81.5 | 63.3 | 95.0 | 15.2 | 2.1 |
| 4 | 50 | 2.5 | 38.0 | 83.4 | 100.0 | 20.0 | 8.1 |
| 5 | 50 | 5.0 | 62.0 | 71.7 | 100.0 | 17.2 | 4.9 |
| 6 | 50 | 7.5 | 75.8 | 62.4 | 95.9 | 15.0 | 3.1 |
| 7 | 55 | 2.5 | 37.1 | 84.4 | 99.6 | 20.3 | 8.2 |
| 8 | 55 | 5.0 | 75.5 | 58.8 | 92.1 | 14.1 | 2.9 |
| 9 | 55 | 7.5 | 59.7 | 68.2 | 96.0 | 16.4 | 5.6 |

¹Total DP2 includes any newly formed DP2 species, including allolactose

### Example 2

Experiment 5 of Example 1 was repeated in a 10 L glass reactor with a top down stir mode. The resulting GOS composition, prior to monosaccharide removal, was analyzed at different time points and summarized in Table 2 (in wt % on total carbohydrates).

TABLE 2

| | | | DP2 | | | | | | |
|---|---|---|---|---|---|---|---|---|---|
| | | | | | DP2 | | | | |
| | | | | | other | | | | |
| | | | | Total | than | | | | |
| | Gal | Glc | Lactose | DP2 | lactose | DP3 | DP4 | DP5 | DP6 |
| T = 0 h | 2.2 | 18.3 | 13.2 | 40.0 | 26.8 | 25.1 | 12.2 | 2.0 | 0.2 |
| T = 1 h | 4.6 | 21.5 | 8.6 | 33.2 | 24.6 | 25.8 | 12.5 | 2.2 | 0.2 |
| T = 2 h | 6.7 | 23.7 | 5.5 | 27.5 | 22.0 | 25.6 | 12.7 | 2.3 | 0.2 |
| T = 3 h | 8.3 | 25.5 | 3.6 | 24.2 | 20.6 | 25.4 | 12.1 | 2.3 | 0.3 |
| T = 4 h | 9.5 | 26.7 | 2.4 | 22.9 | 20.6 | 25.1 | 13.1 | 2.5 | 0.3 |
| After inactivation | 9.5 | 27.2 | 2.1 | 23.1 | 21.0 | 24.6 | 12.8 | 2.5 | 0.4 |

After inactivation, the gal-β1,2-glc and gal-β1,3-glc content was 16.8 wt %, based on total carbohydrate content.

### Example 3

Example 2 was repeated at 1,000 L scale. The reaction was performed for 3 hours. The resulted crude HP-GOS was subjected to SSMB in order to remove the monosaccharides.

The GOS composition at different stages in the process (in wt % on total carbohydrates) is presented in Table 3. The HP GOS extract is the SSMB side stream containing the monosaccharides; the raffinate is the product resulting from the SSMB.

Table 4 shows the oligosaccharide distribution in the HP GOS raffinate, after standardization of the oligosaccharide to 100%.

As shown in these tables, a high purity GOS composition was prepared having an oligosaccharide content of 93 wt %, a lactose content of 5.2%, and a DP2 content (other than lactose) of 27.6%, all based on total carbohydrates. The retention of DP2 species other than lactose was 83% after lactose hydrolysis and 78% after SSMB.

TABLE 3

| | | | DP2 | | | | | | | |
|---|---|---|---|---|---|---|---|---|---|---|
| | | | | DP2 | | | | | | |
| | | | | other | | | | | | |
| | | | | than | Total | | | | | |
| | Gal | Glc | Lactose | lactose | DP2 | DP3 | DP4 | DP5 | DP6 | DP7+ |
| Parent | 2.2 | 18.3 | 13.2 | 26.2 | 39.4 | 25.1 | 12.2 | 2.0 | 0.2 | |
| GOS | | | | | | | | | | |
| After | 4.8 | 30.4 | 3.2 | 20.4 | 23.6 | 24.8 | 13.0 | 2.8 | 0.3 | 0.3 |
| enzymatic | | | | | | | | | | |
| hydrolysis | | | | | | | | | | |
| HP GOS | 11.2 | 73.2 | 0.8 | 7.7 | 8.5 | 4.8 | 2.0 | 0.4 | 0.0 | 0.0 |
| extract | | | | | | | | | | |
| HP GOS | 0.1 | 1.1 | 5.2 | 27.6 | 32.8 | 36.7 | 19.2 | 4.1 | 0.4 | 0.4 |
| raffinate³ | | | | | | | | | | |

³final HP GOS syrup before concentrating to 75% dry matter content

TABLE 4
oligosaccharide distribution of the produced GOS syrup,
based on total oligosaccharide content

| | DP2 | | | | | |
|---|---|---|---|---|---|---|
| | other | | | | | |
| | than | | | | | |
| | lactose | DP3 | DP4 | DP5 | DP6 | DP7+ |
| Parent GOS | 33.1 | 40.3 | 21.1 | 4.5 | 0.5 | 0.5 |
| After | 51.9 | 32.1 | 13.3 | 2.4 | 0.2 | 0.1 |
| enzymatic | | | | | | |
| hydrolysis | | | | | | |
| HP GOS | 31.3 | 41.5 | 21.7 | 4.6 | 0.4 | 0.4 |
| extract | | | | | | |
| HP GOS | 39.7 | 38.0 | 18.5 | 3.1 | 0.3 | 0.5 |
| raffinate | | | | | | |

After inactivation, the gal-β1,2-glc and gal-β1,3-glc content was 16.7 wt %, based on total carbohydrate content.

1: Process for reducing the lactose content of a galacto-oligosaccharide composition, comprising the steps of:
a) providing an aqueous solution of a galacto-oligosaccharide composition with a lactose content of 10-30 wt %, based on total carbohydrate weight, and a weight ratio of (gal-β1,2-glc+gal-β1,3-glc):lactose in the range 1.5:1 to 3.0:1, said solution having a dry matter content of at most 54 wt %,
b) adding to this solution a lactase enzyme,
c) conducting an enzymatic reaction at 30-50° C. to allow hydrolysis of lactose towards monosaccharides, the reaction being conducted until the lactose content of the solution is reduced to at most 6 wt % of lactose, based on total carbohydrate content,
d) removing monosaccharides from the solution obtained in step c).

2: Process according to claim 1 wherein the enzymatic reaction of step c) is conducted until the lactose content of the solution is reduced to at most 5.5 wt %, preferably at most 5.0 wt %, more preferably 3.0-5.0 wt %, even more preferably 3.5-5.0 wt %, and most preferably 4.0-5.0 wt %, based on total carbohydrate content.

3: Process according to claim 1, wherein the monosaccharides are removed by nanofiltration, simulated moving bed chromatography (SMB), or sequential simulated moving bed chromatography (SSMB), preferably by sequential simulated moving bed chromatography.

4: Process according to claim 1, resulting in a galacto-oligosaccharide composition with a lactose content of less than 6 wt %, preferably less than 5.5 wt %, more preferably less than 5.0 wt %, and most preferably 3.0-5.0 wt %, based on carbohydrate content.

5: Process according to claim 1 wherein the aqueous solution of a galacto-oligosaccharide composition used in step a) has been obtained by conversion of lactose using beta-galactosidase derived from Bacillus circulans, Kluyveromyces fragilis, Sporobolomyces singularis, Lactobacillus fermentum, Papiliotrema terrestris, Bifidobacterium bifidum, or Lactobacillus bulgaris, preferably from Bacillus circulans or Papiliotrema terrestris.

6: Process according to claim 1 wherein the lactase enzyme added in step b) is selected from lactase enzymes derived from Kluyveromyces lactis, Lactobacillus bugaricus, or Bifidobacterium bifidum, preferably from Kluyveromyces lactis.

7: Galacto-oligosaccharide composition obtainable by the process of claim 1 and comprising, based on carbohydrate content, 10-40 wt % of the disaccharides gal-β1,2-glc and gal-β1,3-glc, less than 6 wt % lactose, and less than 6 wt % of monosaccharides.

8: Galacto-oligosaccharide composition according to claim 7 comprising less than 5.5 wt %, even more preferably less than 5 wt %, more preferably less than 4.5 wt %, and most preferably less than 4 wt % of monosaccharides, based on carbohydrate content.

9: Nutritional composition comprising the galacto-oligosaccharide composition of claim 7.

10: Food supplement comprising the galacto-oligosaccharide composition of claim 7.
